(19) United States
(12) Patent Application Publication
(10) Pub. No.: US 2008/0187588 A1
(43) Pub. Date: Aug. 7, 2008

(54) Formulations With Controlled Release Of Active Ingredient

(75) Inventors: Susanne Zuleger, Koln (DE); Peter Serno, Bergisch Gladbach (DE); Helmut Haning, Wuppertal (DE); Kerstin Pauli, Dortmund (DE); Roland Heinig, Wuppertal (DE)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/579,925

(22) PCT Filed: May 10, 2005
(86) PCT No.: PCT/EP05/05023

§ 371 Date: Nov. 26, 2007

(30) Foreign Application Priority Data
May 11, 2004 (DE) 102004023069.2

(51) Int. Cl. A61K 9/26; A61K 31/4985; A61P 15/10; A61K 9/00

(52) U.S. Cl. 424/469; 514/250; 424/400; 424/484

(57) ABSTRACT

The present invention relates to novel pharmaceutical dosage forms with controlled release of active ingredient which comprise the PDE 5 inhibitor vardenafil and/or pharmaceutically acceptable salts, hydrates, solvates and/or polymorphic forms thereof as active ingredient, and to the production thereof. The invention further relates to the use of these novel pharmaceutical dosage forms as medicaments, and to their use for producing medicaments for the treatment and/or prevention of disorders in humans and animals.

The present invention relates to novel pharmaceutical dosage forms with controlled release of active ingredient which comprise the PDE 5 inhibitor vardenafil and/or pharmaceutically acceptable salts, hydrates, solvates and/or polymorphic forms thereof as active ingredient, and to the production thereof. The invention further relates to the use of these novel pharmaceutical dosage forms as medicaments, and to their use for producing medicaments for the treatment and/or prevention of disorders in humans and animals.

The PDE 5 inhibitor vardenafil is the compound of the formula (I) having the systematic name {2-ethoxy-5-[(4-ethyl-1-piperazinyl)sulphonyl]phenyl}-5-methyl-7-propylimidazo[5,1-f]triazin-4(3H)-one:

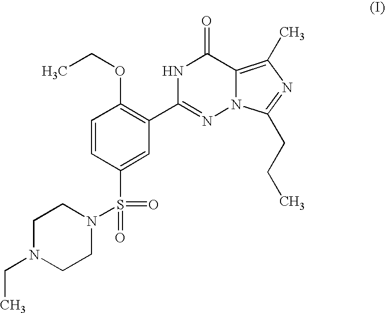

The intracellular cGMP level is controlled by the interplay of synthesis by NO-activated guanylate cyclase on the one hand and degradation by phosphodiesterases (PDEs) on the other hand. PDE 5 in corpus cavernosum tissue of the penis is mainly responsible for controlling the cGMP level which is important for erection.

The NO/cGMP system plays a crucial role in the haemodynamic process of erection. Inhibition of the cGMP-degrading enzyme PDE 5 is effective in particular in situations with elevated NO levels, especially in association with sexual stimulation. Owing to this situation, long-lasting plasma levels of a PDE 5 inhibitor may display an effect against sexual dysfunction each time sexual stimulation occurs. In other diseases too, it is possible for a long-lasting exposure of a PDE 5 inhibitor to lead to an improved therapeutic effect, a reduced fluctuation in the plasma levels, a reduction in the dose to be administered and/or to reduced side effects.

The present invention relates to novel pharmaceutical dosage forms of the PDE 5 inhibitors vardenafil, its salts, hydrates, solvates, polymorphic forms and in particular of the hydrochloride trihydrate, which are distinguished by controlled release of the active ingredient.

Vardenafil and its preparation and use are described for example in WO 99/24433, WO 02/50076, WO 02/089808 and WO 03/011262.

Dosage forms with controlled release of active ingredient are known in principle in the art. Dosage forms with controlled release of active ingredient which comprise cGMP PDE 5 inhibitors are by contrast only little known. Although WO 00/24383 claims pharmaceutical formulations of PDE 5 inhibitors with controlled release, vardenafil differs substantially from these PDE 5 inhibitors because of specific physicochemical and pharmacokinetic properties. It is particularly critical in particular that the absolute oral bioavailability of vardenafil, which is subject to a considerably greater first-pass effect than other cGMP PDE 5 inhibitors such as, for example, sildenafil, is very low, and that the solubility of vardenafil shows a very pronounced pH-dependence.

The prior art to date includes only the use of vardenafil as rapid-release tablet for acute treatment of erectile dysfunction. The use of vardenafil from medicinal forms with controlled release of active ingredient is unknown and has not to date been considered for various reasons.

On the one hand, the medicinal forms of the PDE 5 inhibitor vardenafil which have been used to date and are exclusively rapid-release have satisfied, through their rapid onset of action and their limited, relatively short duration of action, the target profile which has existed to date of a treatment based on need, without unnecessarily long exposure of the patient to the substance.

On the other hand, the worry has been that there might be an increased occurrence of unwanted side effects such as, for example, backache on longer-lasting exposure, as have been observed to be increased on use of PDE 5 inhibitors with a long elimination half-life.

It has thus not appeared possible to date to employ vardenafil as slow-release medicinal form without adversely affecting the reliability and safety of the medicament therapy.

In addition, because of the physicochemical and pharmacokinetic properties of vardenafil, it salts, hydrates and solvates it has been regarded as impossible to develop and use a slow-release medicinal form of vardenafil. The solubility of vardenafil, its salts, hydrates and solvates is extremely pH-dependent, e.g. the solubility of vardenafil hydrochloride trihydrate in 0.1 N hydrochloric acid is 65 mg/ml (soluble), in 0.15 M phosphate buffer of pH 4 is 0.87 mg/ml (very slightly soluble) and in 0.15 M phosphate buffer of pH 7 is 0.03 mg/ml (practically insoluble). This great pH-dependence represents a hindrance in particular to the development of pharmaceutical dosage forms with controlled release of active ingredient for oral use, because the medicinal form is exposed on passage through the gastrointestinal (GI) tract to media with pH values varying greatly from about pH 1 to pH 7.5. In order to achieve a sufficient bioavailability from a slow-release medicinal form, a substance must be absorbed if possible over the entire gastrointestinal tract. However, in the case of vardenafil, a very narrow absorption window was to be expected because the substance is practically insoluble in lower sections of the GI tract, and it was thus necessary to assume that the active ingredient precipitates in lower sections of the small intestine and in the colon, and therefore is not absorbed from substantial regions of the GI tract. It is additionally known that vardenafil is 85% degraded in the first pass and therefore has a very low absolute oral bioavailability of only 15%, which is distinctly lower than the absolute oral bioavailability of other cGMP PDE 5 inhibitors. A slowing of the release of active ingredient usually leads in the case of substances subject to a very large first-pass effect to complete loss of the oral bioavailability, because the slow rise in level of the active ingredient means that the concentrations in the portal vein blood remain so low that the capacity of the metabolizing liver enzymes is sufficient for complete degradation.

For these reasons, vardenafil was to be expected to have a narrow absorption window and an inadequate bioavailability on use in dosage forms which release the active ingredient in controlled fashion over a relatively large region of the GI tract, so that the development of such a formulation appeared virtually impossible.

One theoretical possibility for solving the problem of inadequate bioavailability of substances with a narrow absorption window and preferential absorption in the upper sections of the GI tract is to prolong the residence time of a medicinal form in the upper GI tract. Numerous attempts have been made to develop such dosage forms for other active ingredients, and diverse principles intended to prolong the residence time in the stomach are now known. For example, U.S. Pat. No. 6,306,439 describes the use of swelling systems which are intended to remain in the stomach through swelling and volume expansion, whereas EP 0415671 claims systems with a specific geometry and size. Bioadhesive properties are on the other hand intended to be utilized, for example in the formulations described in WO 03/051304, for prolonging the transit time. Floating medicinal forms of very low density represent a further principle, as described for example in U.S. Pat. No. 5,626,876, but formulations with high density are also claimed for prolonging the residence time in the stomach, as for example in EP 0526862. However, it has not to date been possible to show the success of such systems in practice. Especially when administered in the fasting state, such systems do not show the desired effect of a prolonged residence time in the stomach, because the medicinal forms are emptied from the stomach without noticeable delay through the housekeeper waves.

It was for these reasons desired to develop a medicinal formulation for vardenafil, its salts, hydrates, solvates and polymorphic forms through which the prior art problems described above are overcome.

It has now surprisingly been possible to develop dosage forms which release the active ingredient vardenafil in controlled fashion over a prolonged period throughout the gastrointestinal tract. It was therefore possible to find medicament formulations with particular release profiles through which the prior art problems described above can be overcome. It is crucial in this connection that an average release rate between 80% in 2 hours and 80% in 24 hours is maintained.

It was possible to show in clinical studies on numerous formulations with these specific release profiles that the substance is also absorbed from deep sections of the GI tract on use of these dosage forms according to the invention.

After it had originally been assumed that a rapid-release formulation with limited exposure time represents the optimal dosage form for vardenafil for the therapy of erectile dysfunction, it has now been found that a longer exposure time has distinct advantages. A prolonged exposure with vardenafil on use of a medicament with controlled release of active ingredient makes it possible to prolong substantially the time window in which improved sexual functions can be achieved, so that sexual activities are made possible over a prolonged period, e.g. for up to 24 h after administration of the vardenafil-containing medicament. A distinctly improved flexibility and spontaneity in the patient's sex life is thus achieved, and a better result of therapy and an increase in patient satisfaction are attained.

In addition, dosage forms with controlled release of the PDE 5 inhibitor vardenafil active ingredient are also suitable for the therapy of other, new indications and show substantial advantages over the rapid-release medicinal forms of the prior art. The use of the novel medicinal forms with controlled release of active ingredient has made it possible to achieve substantially more constant blood levels and avoid the occurrence of blood level peaks, thus improving for example the therapeutic efficacy and reducing the frequency and intensity of unwanted side effects. In addition, the use of such dosage forms allows the frequency of administration to be reduced and thus leads to improved acceptance and compliance by the patient.

The clinical studies astonishingly also disclose that, contrary to the previous expectation based on the prior art, it is possible to prolong exposure without the occurrence of an increase in side effects, an adverse effect on reliability and safety of the therapy.

The invention thus relates to novel pharmaceutical dosage forms which comprise vardenafil and/or pharmaceutically acceptable salts, hydrates, solvates and/or polymorphic forms thereof as active ingredient and have an average release rate of between 80% in 2 hours and 80% in 24 hours.

To ascertain the initial release and the average release rate according to the definition of the invention, the release of active ingredient from the dosage forms according to the invention is tested in the paddle apparatus “Apparatus 2” of USP 28-NF23 (The United States Pharmacopoeia USP 28 2005). The release medium used is 900 ml of a phosphate buffer of pH 6.8 with 0.1% (m/V) sodium lauryl sulphate (preparation of 1 liter of this medium: 2.747 g of disodium hydrogen orthophosphate dihydrate, 0.475 g of citric acid monohydrate and 10 g of 10% (m/m) sodium lauryl sulphate solution are dissolved in deionized water and made up to 1000 ml. If necessary, the pH is adjusted to 6.8±0.05 with sodium hydroxide or ortho-phosphoric acid). The release is carried out using sinkers at a temperature of 37±0.5° C. and with a speed of revolution of the paddle of 75 revolutions per minute (rpm). Samples are taken from the release medium through a filtration unit which must ensure that concomitant substances are removed, and the amount of active ingredient dissolved therein is determined by HPLC with UV-VIS detection. The amount of active ingredient determined in this way is converted into percent by mass of the amount of active ingredient employed. The average release rate in the context of the present invention is defined via the time until the release of active ingredient reaches 80%, whereas the initial release describes the percentage release of active ingredient after 30 minutes.

The dosage forms according to the invention with controlled release of active ingredient preferably have an average release rate of 80% in the time interval between 3 and 20 hours (80% in 3 hours and 80% in 20 hours).

In a particularly preferred embodiment of the medicament formulations with controlled release of active ingredient of the present invention, the formulation has an average release rate of 80% in the period from 3 and 18 hours and an initial release not exceeding 65% of the active ingredient in the first 30 minutes of release.

The dosage forms according to the invention with controlled release of active ingredient can be formulated so that a relative low initial release of 0 to 30% in the first 30 minutes or a relative high initial release of 30 to 60% of the medicinal substance in the first 30 minutes of medicinal substance release is achieved.

In a preferred embodiment of the dosage forms with controlled release of active ingredient of the present invention with an average release rate of 80% in the period from 4 to 18 hours, this has a relatively low initial release of 0 to 25% in the first 30 minutes of release.

Another preferred configuration of the medicament formulations with controlled release of active ingredient has an average release rate of 80% in the period from 3 to 16 hours and is distinguished by a relatively high initial release of 35 to 60% in the first 30 minutes of release of active ingredient.

Dosage forms with controlled release of active ingredient of this invention refers to all formulations in which the release of active ingredient is modified so that it takes place with a lower delivery rate than from rapid-release medicinal forms such as, for example, a conventional tablet or capsule.

Dosage forms with controlled release of active ingredient of the present invention also include formulations with delayed release in which the delivery of the active ingredient is modified so that the release starts at a later time than with a conventional rapid-release medicinal form. The subsequent release from a delayed-release medicinal form may also take place in controlled fashion with a reduced release rate.

The dosage forms according to the invention with controlled release of active ingredient also include formulations with pulsatile release, where the delivery of active ingredient takes place intermittently at various times or at particular sites in the gastrointestinal tract, and formulations in which different principles of controlled delivery of active ingredient are combined.

The dosage forms of this invention additionally include also medicament formulations which comprise part of the active ingredient in rapid-release form and a further part of the active ingredient in controlled-release form.

A particular aspect of the present invention is represented by dosage forms with controlled release of active ingredient which comprise acids, bases, buffer substances and/or substances with pH-dependent solubility, such as, for example, polymers resistant to gastric juice, as additives.

The novel formulations with controlled release behaviour can be administered by various routes. Oral administration is particularly preferred, but other administration routes are also possible, e.g. buccal, sublingual, inhalational, ocular, transdermal or rectal administration or use in the form of an implant.

It is possible to employ solid, semisolid or liquid formulations with controlled release behaviour. Solid dosage forms are preferred. The medicament formulations according to the invention may comprise the active ingredient in dissolved, suspended and/or solid, amorphous or crystalline form. The active ingredient can be employed in various particle sizes, e.g. in unground, ground or in micronized form, to produce the dosage forms according to the invention with controlled release of active ingredient.

The dosage forms described above with controlled release of active ingredient are for example in the form of active ingredient-containing particles such as, for example, pellets, granules, microcapsules, tablets, extrudates or as active ingredient crystals which are coated with a diffusion-controlling membrane. These diffusion-controlled systems are preferably multiparticulate, i.e. they preferably consist of a large number of coated cores such as, for example, of neutral pellets onto which a mixture of the active ingredient with a conventional binder and thickener, where appropriate together with conventional excipients and carriers, as defined below for example, is applied and subsequently coated with a diffusion coating which may comprise plasticizers and other excipients. The diffusion-controlled systems according to the invention may additionally consist of homogeneous active ingredient-containing cores which are produced for example by granulation, rotor granulation, fluidized bed agglomeration, tableting, wet extrusion or melt extrusion, where appropriate with spheronization, and are coated with a diffusion coating which may comprise plasticizers and other excipients. In a preferred embodiment of this invention, the active ingredient-containing particles comprise excipients such as, for example, acids or buffer substances which modify the pH and thus contribute to reducing the dependence of the release of active ingredient on the pH of the release medium. In a further preferred embodiment of this invention, the diffusion-controlled membrane comprises excipients which, through their pH-dependent solubility, influence the permeability of the membrane at different pH values and thus help to minimize the pH-dependence of the release of active ingredient.

The binders and thickeners preferably used in the production of coated neutral pellets (e.g. consisting of sucrose, microcrystalline cellulose, citric acid) are hydroxypropylmethylcellulose (HPMC) and polyvinylpyrrolidone (PVP). It is likewise possible to employ other naturally, synthetic or partially synthetic polymers such as, for example methylcellulose (MC), hydroxy-propylcellulose (HPC), other hydroxyalkylcelluloses and hydroxyalkylmethylcelluloses, carboxy-methylcelluloses and salts thereof, polyacrylic acids, polymethacrylates, gelatin, starch or starch derivatives.

Binders and fillers employed for the production of active ingredient pellets, active ingredient-containing particles and (mini)tablets, by granulation, fluidized bed agglomeration, wet extrusion, tableting are for example cellulose, microcrystalline cellulose, cellulose derivatives such as, for example, HMPC, HPC and low-substituted hydroxypropylcellulose (L-HPC), dicalcium phosphate, lactose, PVP and sucrose.

Melt extrusion pellets are produced by incorporating the active ingredient into thermoplastic excipients such as, for example, HPC, HPMC, ethylcellulose, hydroxypropymethylcellulose acetate succinate (HPMCAS), PVP, vinylpyrrolidone/vinyl acetate copolymer, polyethylene glycol, polyethylene oxide, polymethacrylates, polyvinyl alcohols (PVA), partially hydrolysed polyvinyl acetate (PVAc), polysaccharides (e.g. alginic acid, alginates, galactomannans) waxes, fats and fatty acid derivatives.

In a preferred embodiment of this invention, pH-modifying substances such as, for example, acids, bases and buffer substances are incorporated into the active ingredient-containing core. Addition of these substances makes it possible to reduce markedly the pH-dependence of the release of vardenafil and its salts, hydrates, solvates. Examples of suitable excipients which modify the pH in the active ingredient-containing cores are: adipic acid, malic acid, L-arginine, ascorbic acid, aspartic acid, benzenesulphonic acid, benzoic acid, succinic acid, citric acid, ethanesulphonic acid, 2-hydroxyethanesulphonic acid, fumaric acid, gluconic acid, glucuronic acid, glutamic acid, potassium hydrogen tartrate, maleic acid, malonic acid, methanesulphonic acid, toluenesulphonic acid, trometamol, tartaric acid. Citric acid, succinic acid, tartaric acid, potassium hydrogen tartrate are preferably employed.

Particularly suitable for producing the diffusion coating are ethylcelluloses, e.g. as aqueous dispersion commercially available under the name Aquacoat® or Surelease®, and polymethacrylates such as, for example, Eudragit® NE, Eudragit® RS and RL. However, other materials such as, for example, cellulose acetate and cellulose acetate butyrate can also be employed as film-forming diffusion-controlling polymers.

In a preferred embodiment of this invention, the diffusion coating comprises, besides the diffusion-controlling polymer, also excipients with pH-dependent solubility such as, for example, polymers resistant to gastric juice, such as cellulose phthalates, especially cellulose acetate phthalate and hydroxypropylmethylcellulose phthalate, cellulose succinates, especially cellulose acetate succinate and hydroxypropylmethylcellulose acetate succinate or polymethacrylates (e.g. Eudragit® L). Addition of these substances makes it possible to speed up the release of vardenafil and its salts, hydrates, solvates at higher pH values (e.g. pH 4.5 and pH 6.8) and thus to reduce the pH-dependence of the release of active ingredient. These substances with pH-dependent solubility are added in a proportion of from 0 to 60% (m/m), preferably 10 to 50% (m/m) based on the mass of the film.

Examples of plasticizers used are citric acid derivatives (e.g. triethyl citrate, tributyl citrate, acetyl triethyl citrate), phthalic acid derivatives (e.g. dimethyl phthalate, diethyl phthalate, dibutyl phthalate), benzoic acid and benzoic esters, other aromatic carboxylic esters (e.g. trimellithic esters), aliphatic dicarboxylic esters (e.g. dialkyl adipates, sebacic esters, in particular diethyl sebacate, tartaric esters), glycerol monoacetate, glycerol diacetate or glycerol triacetate, polyols (e.g. glycerol, 1,2-propanediol, polyethylene glycol of varying chain length), fatty acids and derivatives (e.g. glycerol monostearates, acetylated fatty acid glycerides, castor oil and other natural oils, Miglyol) and fatty acid alcohols (e.g. cetyl alcohol, cetylstearyl alcohol). The nature and amount of the plasticizer are chosen so that the above-defined release according to the invention and the necessary stability of the medicinal forms is achieved. The proportion of the plasticizer is expediently from 0 to 50% (n/m), preferably 0 to 35% (m/m), particularly preferably 0 to 25% (m/m) based on the mass of the film.

In order to prevent adhesion of the coated particles during production and in the finished product, it is possible to add to the coating so-called antistick agents such as, for example, talc, magnesium stearate, glycerol monostearate and Aerosil. The proportion of these antistick agents depends on the used polymer and plasticizer or proportion of plasticizer and is normally from 0 to 50% (m/m) of the total mass of the coating film.

The release rate according to the invention is controlled by the coating composition and the thickness of the coating layer. So-called “pore formers” can be put into the coating or into the particle to be coated as additions which increase the permeability of the film. The pore formers employed are soluble polymers such as, for example, polyethylene glycols, PVP, PVA, HPMC, HPC, hydroxyethylcelluloses (HEC), MC, carboxymethylcelluloses or their salts, dextrins, maltodextrins, cylcodextrins, dextrans or other soluble substances such as, for example, urea, salts (sodium chloride, potassium chloride, ammonium chloride, etc.), sugars (sucrose, lactose, glucose, fructose, maltose etc.), sugar alcohols (mannitol, sorbitol, xylitol, lactitol, etc.). Based on the mass of the diffusion film, from 0 to 50% (m/m), preferably 0 to 35% (m/m), particularly preferably 0 to 20%, of pore-former are employed.

Excipients with pH-dependent solubility which may be constituents of the diffusion film are, for example, polymers resistant to gastric juice, such as cellulose phthalates, especially cellulose acetate phthalate and hydroxypropylmethylcellulose phthalate, cellulose succinates, especially cellulose acetate succinate and hydroxypropylmethylcellulose acetate succinate and polymethacrylates (e.g. Eudragit® L).

Based on the total mass, the described diffusion-controlled medicinal forms consist of 0.5 to 50% (n/m), preferably 2 to 40% (m/m), active ingredient (calculated as vardenafil), 10 to 95% (m/m) binder/filler or thermoplastic excipient in the case of melt extrusion pellets, and 5 to 50% (m/m), preferably 5 to 40% (m/m), particularly preferably 5 to 30% (m/m), diffusion coating and they may comprise further additives (pH-modifying substances, other pharmaceutical conventional excipients).

The diffusion coating or diffusion layer comprises, based on the amount of coating, from 40 to 100% (m/m), preferably 50 to 100% (m/m), film former (film-forming diffusion-controlling polymers and, where appropriate, polymers resistant to gastric juice), 0 to 50% (m/m), preferably 0 to 35% (n/m), particularly preferably 0 to 25%, plasticizer and 0 to 50% (n/m), preferably 0 to 35% (m/m), particularly preferably 0 to 20% (m/m), pore former (water-soluble polymers and other water-soluble substances). The coating may additionally comprise antistick agents, 0 to 50% (m/m), based on the film mass, and other additives (pigments, colorants, surfactants, emulsifiers, other pharmaceutical conventional excipients).

A further aspect of the present invention are coated dosage forms which comprise one or more swellable excipients which, on penetration of liquid through the membrane, swell greatly and, through the swelling and volume expansion, cause the coating to split. The splitting of the coating makes it possible for the medicinal substance to be released from the dosage form, usually in pulsatile form. Swellable excipients which these formulations may comprise are, for example, polyvinylpyrrolidones, crospovidones, crosslinked sodium carboxymethylcellulose, crosslinked sodium carboxymethylstarch, polyethylene oxides, polymethyacrylates, low-substituted hydroxypropylmethylcellulose (L-HPC). Examples of suitable coating materials are cellulose acetate, ethylcellulose and polymethacrylates.

The described coated, diffusion-controlled or pulsatile formulations can be employed directly and unmodified as medicinal form. However, they may also be further processed, where appropriate with addition of excipients, to the final dosage form (e.g. capsule, tablet, sachet formulation). In order to achieve a desired release profile it is also possible to combine different coated formulations in one medicinal form, and administration of an initial dose can take place for example by combination with rapid-release formulation particles, e.g. uncoated pellets, granules or powder.

In a further embodiment of the dosage forms according to the invention with controlled release there is use of formulations which include the active ingredient in a matrix. These so-called matrix formulations release the active ingredient by diffusion and/or erosion. These formulations are preferably in the form of a tablet or in the form of a plurality of tablets which may be for example encapsulated. The tablets may be coated. Such matrix formulations are produced for example by mixing the ingredients and direct tableting or by dry or wet granulation with subsequent tableting.

The mass ratio of active ingredient to the total mass of the matrix formulation in these novel formulations is in the range from 1:1 to 1:200, preferably in the range from 1:2 to 1:40.

The proportionate amount of the matrix former is preferably in the range from 10 to 70% (m/m) of the mass of the formulation.

Matrix formers which can be employed are water-soluble, water-swellable or water-insoluble substances. The novel formulations preferably comprise one or more water-swellable polymers.

Preference is additionally given to medicinal preparations in the context of this invention which comprise water-soluble, hydrogel-forming polymers, these polymers having a nominal viscosity of at least 15 cP, preferably at least 50 cP (measured as 2% strength aqueous solution at 20° C.).

Water-soluble or water-swellable matrix-forming polymers preferably employed are hydroxy-propylmethylcelluloses (HPMC), hydroxyethylmethylcelluloses, hydroxypropylcelluloses (HPC), hydroxyethylcelluloses methylcelluloses (MC), ethylcelluloses, other alkylcelluloses, hydroxy-alkylcelluloses and hydroxyalkylmethylcelluloses, sodium carboxymethylcelluloses (NaCMC), alginates, galactomannans such as, for example, guar and carob flour, xanthans, polyethylene oxides, polyacrylic acids, polymethacrylic acids, polymethacrylic acid derivatives, polyvinyl alcohols (PVA), partially hydrolysed polyvinyl acetate (PVAc), polyvinylpyrrolidone (PVP), agar, pectin, gum arabic, tragacanth, gelatin, starch or starch derivatives and mixtures of these substances.

The use of HPMC is particularly preferred.

In this connection, the matrix formulations according to the invention should preferably comprise at least 10% of a hydroxypropylmethylcellulose type whose nominal viscosity (measured as 2% strength aqueous solution at 20° C.) is at least 15 cP, preferably at least 50 cP. HPMC types preferably used have a degree of substitution of methoxy groups of 16.5-30%, particularly preferably 19-30%, and a degree of substitution of hydroxypropoxy groups of 4-32%, particularly preferably 4-12%.

It is furthermore possible to employ water-insoluble substances as matrix formers in the matrix formulations according to the invention, e.g. unsaturated or saturated/hydrogenated fatty acids and their salts, esters or amides, fatty acid mono-, di- or triglycerides, waxes, ceramides, cholesterol derivatives and mixtures of these substances.

The formulations of the present invention may comprise conventional tableting aids such as, for example, colloidal silicon dioxide (Aerosil®), magnesium stearate, talc, PVP, lactose or microcrystalline cellulose. These are present in the case of lactose and microcrystalline cellulose normally in an amount of from 10 to 50%, in the case of Mg stearate expediently in an amount of from 0.5 to 3% and in the case of Aerosil expediently in an amount of from 0.1 to 2%, based on the mass of the tablet.

In a particularly preferred embodiment of this invention, substances which control the pH in the matrix are incorporated into the matrix. The addition of such pH-modifying excipients and/or the addition of substances which dissolve or are dissolved out of the matrix as the pH increases, and thus increase the porosity or permeability of the matrix and/or promote erosion of the matrix, makes it possible to achieve a virtually pH-independent release for these preferred embodiments of the present invention.

Examples of suitable excipients which can be added to the matrix formulations according to the invention to achieve release which is as far as possible pH-independent are the following substances: adipic acid, malic acid, L-arginine, ascorbic acid, aspartic acid, benzenesulphonic acid, benzoic acid, succinic acid, cellulose phthalates, in particular cellulose acetate phthalate and hydroxypropylmethylcellulose phthalate, cellulose succinates, in particular cellulose acetate succinate and HPMCAS, citric acid, ethanesulphonic acid, 2-hydroxyethanesulphonic acid, fumaric acid, gluconic acid, glucuronic acid, glutamic acid, potassium hydrogen tartrate, maleic acid, malonic acid, methanesulphonic acid, polymethacrylates (e.g. Eudragit® types), toluenesulphonic acid, trometamol, tartaric acid. Citric acid, succinic acid, tartaric acid, HPMCAS, and polymethacrylates (e.g. Eudragit® L) are preferably employed. If these excipients are present in the matrix formulations according to the invention, they are typically added in a proportion of from 10 to 50% (m/m) based on the total mass of the matrix.

The active ingredient-containing matrix may also have specific geometric shapes with which the release is influenced by the specific geometry and matrix surface. The matrix surface and release surface can be controlled for example by compression to special formats (e.g. annular tablets), and/or by coating partial areas or applying barrier layers by means of a multilayer press.

Formulations with different release properties can be combined for example in multilayer or shell-core tablets to give a medicinal form. Thus, for example, the controlled releases according to the invention with high initial release of active ingredient are achieved by multilayer tablets which include a rapid-release layer, or shell-core tablets with a rapid-release shell, whereas a final acceleration of release (late burst) can be achieved by shell-core tablets with a rapid-release core.

Another configuration of the dosage forms according to the invention with controlled release of active ingredient is characterized in that the active ingredient is incorporated by a melting process into a matrix consisting of one or more physiologically acceptable excipients. The release of active ingredient from these so-called melt extrudates takes place by diffusion and/or erosion. These formulations are preferably in the form of granules, pellets or tablets. The forms obtained by melt extrusion, especially pellets and granules, can be further processed to other medicinal forms, such as, for example, by encapsulation or tableting, where appropriate with addition of pharmaceutically conventional excipients. The melt extrudates according to the invention may additionally be ground and subsequently be employed in this comminuted form for producing other dosage forms such as, for example, matrix tablets. The further processing also includes combining formulations with different medicinal substance release, such as, for example, slow- and rapid-release particles, to give a dosage form. The melt extrudates and/or the medicinal forms produced from melt extrudates can be coated.

The melt extrudates are produced by mixing the active ingredient with at least one fusible physiologically acceptable excipient (carrier) and, where appropriate, further conventional pharmaceutical additives, melting at a temperature in the range from 50 to 250° C., preferably 60 to 200° C., injection moulding or extruding and shaping. It is possible in this case for the mixing of the components to take place either before the melting or during the melting, or part of the components are melted and the other ingredients are admixed to this melt. The mixture of the carrier, the active ingredient and any additives present is thermoformable after melting and can therefore be extruded. Numerous methods are suitable for shaping the mixture, for example, hot granulation, cold granulation, calendering with two moulding rolls, extrusion and shaping of the extrudate while still plastic, e.g. between two belts or rolls, or rounding off for example in an air granulating unit after cutting of the extrudate.

The mass ratio of active ingredient to the total mass of the melt extrudate in these novel formulations is in the range from 1:3 to 1:200, preferably in the range from 1:4 to 1:100.

Examples of suitable thermoplastic carriers, which are preferably swellable or soluble in physiological media, are: polyvinylpyrrolidone (PVP), copolymers of N-vinylpyrrolidone (NVP) and vinyl esters, in particular vinyl acetate, copolymers of vinyl acetate and crotonic acid, partially hydrolysed polyvinyl acetate, polyvinyl alcohol, cellulose esters, cellulose ethers, especially methylcellulose and ethylcellulose, hydroxyalkylcelluloses, especially hydroxypropylcellulose, hydroxyalkylmethylcelluloses, especially hydroxypropylmethylcellulose and hydroxyethylmethyl-cellulose, carboxymethylcelluloses, cellulose phthalates, especially cellulose acetate phthalate and hydroxypropylmethylcellulose phthalate, cellulose succinates, especially cellulose acetate succinate and hydroxypropylmethylcellulose acetate succinate, polyhydroxyalkyl acrylates, polyhydroxyalkyl methacrylates, polyacrylates and polymethacrylates (Eudragit® types), copolymers of methyl methacrylate and acrylic acid, polylactides, polyethylene glycols, polyethylene oxides and polysaccharides such as galactomannans and alginic acid and its alkali metal and ammonium salts.

Preferred thermoplastic excipients for producing the dosage forms according to the invention with controlled release of active ingredient are HPC, PVP, vinylpyrrolidone/vinyl acetate copolymers, polymethyacrylates, especially Eudragit® L and RS PO, HPMCAS, polyethylene glycols, polyethylene oxides and mixtures thereof.

Examples of plasticizing excipients which can be employed to reduce the glass transition temperature of the mixture are propylene glycol, glycerol, triethylene glycol, butanediols, pentanols, such as pentaerythritol, hexanols, long-chain alcohols, polyethylene glycols, polypropylene glycols, polyethylene/propylene glycols, silicones, phthalic acid derivatives (e.g. dimethyl phthalate, diethyl phthalate, dibutyl phthalate), benzoic acid and benzoic esters, other aromatic carboxylic esters (e.g. trimellithic esters), citric acid derivatives (e.g. triethyl citrate, tributyl citrate, acetyl triethyl citrate), aliphatic dicarboxylic esters (e.g. dialkyl adipates, sebacic esters, in particular diethyl sebacate, tartaric esters), glycerol monoacetate, glycerol diacetate or glycerol triacetate, fatty acids and derivatives (e.g. glycerol monostearates, acetylated fatty acid glycerides, castor oil and other natural oils, Miglyol), fatty acid alcohols (e.g. cetyl alcohol, cetylstearyl alcohol), sugars, sugar alcohols and sugar derivatives (e.g. erythritol, isomalt, lactitol, mannitol, maltitol, maltodextrin, xylitol). The concentration of plasticizers is normally from 0 to 30% (m/m), preferably from 0 to 20% (m/m) based on the total mass of the melt extrudate.

The extruded mixture may, besides active ingredient, carrier and, where appropriate, plasticizer, also comprise other pharmaceutically conventional additives, for example lubricants and mould release agents, glidants and flow aids, fillers and adsorbents, stabilizers, radical scavengers, complexing agents, antioxidants, photostabilizers, blowing agents, surfactants, preservatives, colorants, sweeteners and flavourings.

The precondition for suitability of a substance as excipient are exclusively sufficient temperature resistance and physiological tolerability.

The proportion of additives in the total mass of the extrudate may be up to 60% (m/m).

Lubricants and mould release agents may, for example stearic acid and stearates, especially aluminium, calcium and magnesium stearates, calcium behenate, sodium stearyl fumarate, talc, silicones, waxes, and mono-, di- and triglycerides such as, for example, glycerol monostearate, glycerol distearates, glycerol dibehenate, glycero monooleate, glyceryl palmitostearate, be added in an amount of from 0 to 10% (n/m), preferably from 0.5 to 5% (m/m), based on the total mass of the melt extrudate.

Examples of flow aids used are animal and vegetable fats, preferably in hydrogenated form and with a melting point of at least 50° C., waxes (e.g. carnauba wax), mono-, di- and triglycerides (e.g. glycerol monostearate, glycerol distearates, glycerol dibehenate, glycero monooleate, glyceryl palmitostearate), phosphatides, especially lecithin, in a total amount of from 0 to 30% (m/m), preferably 0 to 10% (m/m), based on the total mass of the extrudate.

Examples of fillers employed are substances such as titanium dioxide, aluminium oxide, magnesium oxide, silica and silicates, stearic acid and stearates, cellulose derivatives (e.g. methylcellulose), starch and starch derivatives, sugars, sugar alcohols and sugar derivatives, normally in a proportion of from 0 to 30% (m/m), preferably 0 to 20% (m/m), based on the total mass of the extrudate.

A preferred embodiment of the dosage forms according to the invention with controlled release of active ingredient are melt extrudates which comprise excipients with pH-modifying properties and/or pH-dependent solubility. It is possible through these excipients (for example the acids, bases, buffer substances and polymers resistant to gastric juice which have already been described several times previously) to minimize the pH-dependence of the release of vardenafil and its salts, hydrates, solvates.

In the production of the melt extrudates there may be formation of so-called “solid solutions” in which the active ingredient is in the form of a molecular dispersion in the matrix.

A further configuration of the dosage form according to the invention with controlled release of active ingredient are osmotic medicinal substance release systems. Such osmotic systems are known in principle in the prior art. In this case, the delivery of medicinal substance from the medicinal form is generally based on an osmotic pressure as driving force. A detailed description of osmotic systems is given for example in Verma R. K. et. al. “Osmotic pumps in drug delivery”, Critical Reviews™ in Therapeutic Drug Carrier Systems, 21 (2004) 477-520 and Santus G. et al. “Osmotic drug delivery: a review of the patent literature”, Journal of Controlled Release 35 (1995) 1-21.

The osmotic system as embodiment of the present invention preferably consists of:
a core which comprises the active ingredient, where appropriate a hydrophilic polymeric swelling agent and where appropriate a water-soluble substance to initiate osmosis, and where appropriate further pharmaceutically acceptable excipients,
and a shell which consists of a water-permeable material which is impermeable to the components of the active ingredient-containing core, and has at least one orifice through which the ingredients present in the core can be released.

The material from which the shell is formed for these dosage forms according to the invention with controlled release of active ingredient is semipermeable, i.e. permeable to water, aqueous media and biological fluids and impermeable or very slightly permeable to the components of the core, and suitable for film formation. The selective semipermeable shell material is insoluble in body fluids, does not erode, is not degraded in the GI tract and is excreted unchanged, or it shows bioerosion only towards the end of the release time. Typical materials for producing the shell are known from the literature and described for example in the patents U.S. Pat. No. 3,916,899, U.S. Pat. No. 3,977,404 and EP 0277092. It is possible to use for example acylated cellulose derivatives (cellulose esters) which are substituted once to three times by acetyl groups or once to twice by acetyl groups and by a further acyl radical different from acetyl, e.g. cellulose acetate, cellulose triacetate, cellulose acetate ethylcarbamate, cellulose acetate phthalate, cellulose acetate methylcarbamate, cellulose acetate succinate, cellulose acetate dimethylaminoacetate, cellulose acetate diethylaminoacetate, cellulose acetate ethyl carbonate, cellulose acetate chloroacetate, cellulose acetate ethyl oxalate, cellulose acetate methylsulphonate, cellulose acetate butylsulphonate, cellulose acetate propionate, cellulose acetate octate, cellulose acetate laurate, cellulose acetate p-toluenesulphonate, cellulose acetate butyrate and other cellulose acetate derivatives, and agar acetate and amylose acetate. Also suitable as semipermeable membrane material are ethylcellulose, copolymers of alkylene oxide and alkyl glycidyl ether, polymeric epoxides, polyglycols and polylactic acid derivatives. It is additionally possible to employ mixtures of intrinsically water-insoluble acrylates, e.g. a copolymer of ethyl acrylate and methyl methacrylate. If necessary, the shell may also comprise plasticizers such as, for example, the plasticizing substances already mentioned previously, and other additives such as, for example, pore formers. If required, a photoprotective coating which may consist for example of HPMC or HPC, and of a suitable plasticizer (e.g. polyethylene glycol) and pigments (e.g. titanium dioxide, iron oxide), can be applied to the semipermeable shell. In order to be able to administer an initial dose of the active ingredient, the osmotic system can also be provided with an active ingredient-containing coating from which the active ingredient is rapidly released on contact with the release medium, before the osmotically controlled delivery of active ingredient from the core starts.

Examples of suitable water-swellable polymers which may be present in the core are polyethylene oxides having molecular weights of from 100 000 to 8 000 000 (e.g. Polyox®), xanthan gum, copolymers of vinylpyrrolidone and vinyl acetate, polyvinylpyrrolidones, crospovidones, crosslinked sodium carboxymethylcellulose, crosslinked sodium carboxymethylstarch, low-substituted hydroxypropylmethylcellulose (L-HPC), poly(hydroxyalkyl methacrylate), alginates and galactomannans, and further hydrophilic polymeric swelling agents mentioned in the patents U.S. Pat. No. 3,865,108, U.S. Pat. No. 4,002,173, U.S. Pat. No. 4,207,893, EP 0052917, EP 0277092 and WO 96/40080, and mixtures thereof.

Suitable osmotically active substances which can be added to the core to induce osmosis are in principle all water-soluble, physiologically acceptable substances such as, for example, the water-soluble substances mentioned in the pharmacopoeias and in “Remingtons Pharmaceutical Science”. It is possible to employ in particular water-soluble salts of inorganic and organic acids or nonionic organic substances with high solubility in water, such as, for example, carbohydrates, especially sugars, or amino acids. Some substances which can be incorporated singly or as mixture for inducing osmosis in the core are named by way of example in the following: inorganic salts such as chlorides, sulphates, sulphites, carbonates, bicarbonates, phosphates, hydrogen phosphates and dihydrogen phosphates of the alkali metals and alkaline earth metals such as, for example, sodium, lithium, potassium, calcium or magnesium, organic acids such as adipic acid, ascorbic acid, succinic acid, citric acid, fumaric acid, maleic acid, tartaric acid, benzoic acid, and the alkali metal or alkaline earth metal salts thereof, acetates, pentoses such as, for example arabinose, ribose or xylose, hexoses such as glucose, fructose, galactose or mannose, disacchaarides such as sucrose, maltose or lactose, trisaccharides such as raffinose, sugar alcohols such as mannitol, sorbitol, maltitol, xylitol or inositol, and urea.

Sodium chloride and sodium bicarbonate are particularly preferably used.

The osmotic system may additionally comprise other pharmaceutically conventional additives such as, for example, lubricants and mould release agents, glidants, binders, colorants, thickeners, protective colloids, stabilizers and surfactants.

The osmotic release system according to the invention is produced with the aid of standard techniques such as wet granulation or dry compaction and tableting to produce the active ingredient-containing core and subsequent organic coating.

The shell of the osmotic system has at least one exit orifice through which the active ingredient, where appropriate together with other ingredients of the core, is released. The orifice can be introduced into the shell in various ways, e.g. by punching, mechanical drilling or by means of a laser drill. The term “orifice” also includes bioerodable materials which are dissolved out of the shell on use of this dosage form according to the invention, and thus lead in situ to the formation of exit orifices. The nature and production of the orifices are known in the state of the art and are explained for example in the patents U.S. Pat. No. 3,485,770, U.S. Pat. No. 3,916,899, U.S. Pat. No. 4,063,064 and U.S. Pat. No. 4,088,864.

The release rate according to the invention is primarily adjusted through the composition and thickness of the semipermeable shell, through the nature and amount of the polymeric swelling agent which is present where appropriate, and through the nature and amount of the osmotically active substance which is present where appropriate and serves to induce osmosis.

In a further aspect of the invention, the active ingredient in the formulation may be in the form of an ion exchanger complex.

It is possible for a plurality of particles of the abovementioned formulation principles to be present together in one dosage form (e.g. capsule packed with a plurality of active ingredient-containing matrices). It is additionally possible also for a plurality of the various embodiments (e.g. pellets with diffusion coating and matrix tablet) to be combined in one medicinal form.

The present invention further relates to the combination of formulations with different release properties, e.g. rapid-release and slow-release, in one medicinal form.

The medicinal forms according to the invention may be coated, e.g. in order to achieve photoprotection, to mask the taste or to control the site or time of the onset of medicinal substance release.

The dosage form according to the invention with controlled release of active ingredient is preferably a formulation in which the maximum blood level (cₘₐₓ) after administration is reduced by comparison with a rapid-release medicinal form of the same dosage and with which the mean residence time (MRT) of the medicinal substance in the body is proloned in relation to a rapid-release medicinal form.

The pharmacokinetic parameters AUC, tₘₐₓ, cₘₐₓ and MRT are determined as described in Gibaldi M., Perrier D. “Pharmacokinetics”, 2nd edition. Marcel Dekker, New York, 1982 and in Rowland M., Tozer T. N. “Clinical Pharmacokinetics: Concepts and Applications”, Lea & Febiger, Philadelphia, 1980.

The present invention also includes the use of the novel pharmaceutical dosage forms for producing medicaments which are intended for the treatment and/or prevention of disorders in humans and animals.

It has generally proved advantageous on oral administration in humans to administer an active ingredient dose of about 1 to 100 mg, preferably of about 2 to 50 mg. Incremental dosage of the novel dosage forms is also possible, i.e. with a dose progressively increasing over a lengthy period (for example 2-10 days). Treatment with the novel formulation can also take place on a plurality of consecutive days, for example each day or in another fixed time rhythm.

The novel dosage forms according to the invention are suitable for the prophylaxis and/or treatment of disorders in which an increase in the cGMP concentration is beneficial, i.e. disorders connected with cGMP-regulated processes (usually referred to simply as cGMP-related diseases).

The novel dosage forms of the PDE 5 inhibitor vardenafil with controlled release can be employed in medicaments for the treatment of cardiovascular disorders such as, for example, for the treatment and/or prophylaxis of high blood pressure, neuronal hypertension, stable and unstable angina, peripheral and cardiac vascular disorders, of arrhythmias, for the treatment of thromboembolic disorders and ischaemias such as myocardial infarction, cerebral ischaemias, transient ischaemic attacks, angina pectoris, primary pulmonary hypertension, secondary pulmonary hypertension, pulmonary arterial hypertension, portopulmonary hypertension, hepatopulmonary syndrome, pulmonary hypertension induced by drugs such as amphetamines, interstitial lung disease, pulmonary hypertension associated with HIV, thromboembolic pulmonary hypertension, pulmonary hypertension in children and neonates, pulmonary hypertension induced by atmospheric hypoxia (altitude sickness), COPD, emphysema, chronic asthma, mucoviscidosis-related pulmonary hypertension, right heart failure, left heart failure and global failure, peripheral malperfusions, for preventing restenoses after thrombolysis therapy, percutaneous transluminal angioplasty (PTA), percutaneous transluminal coronary angioplasty (PTCA), bypass, for the treatment of cerebrovascular disorders, for the treatment and/or prophylaxis of disorders of the urogenital system such as prostate hypertrophy, incontinence and in particular for the treatment and/or prophylaxis of erectile dysfunction, premature ejaculation, of benign prostate hyperplasia, of female sexual dysfunction and of female sexual arousal impairment.

In addition, a further embodiment of the invention relates to the use of the novel dosage form of the PDE 5 inhibitor vardenafil with controlled release of active ingredient for producing a medicament for the treatment and/or prophylaxis of impairments of perception, concentration, learning and/or memory, in particular when the impairment is a consequence of dementia. The novel formulations according to the invention are particularly suitable for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (“post stroke dementia”), post-traumatic craniocerebral trauma, general concentration impairments, concentration impairments in children with learning and memory problems, of Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoffs psychosis.

The novel dosage forms of the PDE 5 inhibitor vardenafil can also be employed for the treatment and/or prophylaxis of psoriasis, cancer, bladder disorders, nitrate-induced tolerance, pre-eclampsia, alopecia, pain, sudden loss of hearing, tinnitus or the renal syndrome.

The novel formulations of the PDE 5 inhibitor vardenafil can also be used for the treatment and/or prophylaxis of ocular disorders such as glaucoma, of central retinal or posterior cilliary arterial occlusion, central retinal venous occlusion, optic neuropathy such as anterior ischaemic optic neuropathy and glaucomatous optic neuropathy, and of macular degeneration.

The novel formulations of the PDE 5 inhibitor vardenafil can likewise be used to produce medicaments for the treatment and/or prophylaxis of coronary heart disease, diabetes, insulin resistance, hyperglycaemia, pancreatitis, diabetic gastroparesis, diabetic nephropathy, diabetic neuropathy, diabetic retinopathy, diabetic gangrene, diabetic glomerulosclerosis, diabetic dermopathy, diabetic arthropathy, diabetic cataract, for the treatment of impairments of the peristalsis of stomach and oesophagus, of osteoporosis, female infertility, premature labour, cirrhosis of the liver, acute and chronic renal failure, cystic fibrosis, bronchitis and allergic rhinitis.

The novel formulations of the PDE 5 inhibitor vardenafil can also be employed for the treatment and/or prophylaxis of cardiac ischaemia, for achieving or improving a preconditiong effect, for the treatment of an acute myocardial infarction and of reperfusion damage, specifically following a myocardial infarction, for the treatment of male infertility, of Raynaud's syndrome, of intermittet claudication, of Peyronie's disease, for the treatment of fibrotic disorders, of arteriosclerosis, for improving sperm motility, for the treatment of depression, leukaemia (e.g. of chronic lymphocytic leukaemia), for the treatment of priapism, for the treatment of platelet adhesion and aggregation associated with renal ischaemia, for supporting and promoting liver regeneration following surgical resection of the liver or associated with liver cancer, for inhibiting the contraction of oesophageal muscles (e.g. associated with nutcracker oesophagus or oesophagospasms), for the treatment of achalasia, female infertility and dysmenorrhoea, for the treatment of liver disorders such as, for example, cirrhosis of the liver, for the treatment of lupus, hypertensive systemic lupus erythematosus, scleroderma, for the treatment of multiple sclerosis, rheumatoid arthritis, allergy, autoimmune diseases, osteoporosis, kachexia, polycystic ovary syndrome, inflammatory bowel diseases such as, for example, Crohn's disease and ulcerative colitis, hyperlipidaemia and dyslipidaemia, for promoting growth and improving survival of oocytes, zygotes, embryos or foetuses, for increasing the weight of premature babies, for increasing milk production in mammals, specifically in humans, for the treatment of migraine, incontinence, acute and chronic renal failure, of glomerular disease, of nephritis, tubulointerstitial disorders, glomuleropathy, hair loss, amnesia, disturbances of consciousness, autism, speech disturbances, Lennox syndrome and epilepsy.

In addition, use of the novel formulations according to the invention enhances the effect of substances such as, for example, EDRF (endothelium derived relaxing factor), ANP (atrial natriuretic peptide), of nitro vasodilators and all other substances which increase the cGMP concentration in a different way to phosphodiesterase inhibitors.

The novel dosage forms of the PDE 5 inhibitor vardenafil can also be used in combination with other medicament active ingredients. Preferred examples in this connection are inhibitors of HMG-CoA reductase (e.g. simvastatin, atorvastatin, fluvastatin, rosuvastatin, pravastatin, itavastatin), CETP inhibitors (e.g. torcetrapib, JTT-705), ACE inhibitors (e.g. enalapril, captopril, benazepril, cilazapril, fosinopril, quinapril, lisinopril, ramipril), PPARalpha agonists (e.g. fenofibrate, bezafibrate, GW 590735), PPARgamma agonists (e.g. rosiglitazone), aldose reductase inhibitors, ezetimibe, platelet aggregation inhibitors (e.g. aspirin, clopidogrel, ticlopidine, dipyridamole), thrombin inhibitors (e.g. ximelagatran, melagatran, bivalirudin, clexane), beta-blockers (e.g. propanolol, atenolol), diuretics (e.g. furosemide), insulin and insulin derivatives, and orally active hypoglycaemic active ingredients. Insulin and insulin derivatives include in this connection both insulins of animal, human or biotechnological origin and mixtures thereof. The novel dosage forms of the PDE 5 inhibitor verdenafil can furthermore also be employed in combination with sulphoanylureas (e.g. tolbutamide, glibenclamide, glimepiride, glipizide or gliclazide), biguanide derivatives (e.g. metformin), alpha-glucosidase inhibitors (e.g. miglitol or acarbose), meglitinides (e.g. repaglinide, nateglinide), anti-obesity active ingredients (e.g. orlistat, sibutramine), GPIIb-IIIa antagonists (e.g. tirofiban, abciximab), factor Xa inhibitors (e.g. DX 9065a, DPC 906, JTV 803, BAY 597939), calcium antagonists (e.g. nifedipine, amlodipine, verapamil, diltiazem), antagonists of alpha1 receptors, angiotensin AII antagonists (e.g. candesartan, losartan, valsartan, telmisartan), other PDE 5 inhibitors (e.g. sildenafil, tadalafil) or other active ingredients for the treatment of erectile dysfunction (e.g. apomorphine).

The novel formulations of the PDE 5 inhibitor vardenafil can be produced by using besides the hydrochloride trihydrate and its polymorphic, crystalline and amorphous forms also other physiologically acceptable salts of vardenafil, and vardenafil itself. Physiologically acceptable salts may be salts of vardenafil with inorganic or organic acids. Preference is given to salts with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, lactic acid, benzoic acid, or methanesulphonic acid, ethanesulphonic acid, phenylsulphonic acid, toluenesulphonic acid or naphthalenedisulphonic acid. The use of these salts and their polymorphic, crystalline and amorphous forms, and the use of the polymorphic, crystalline and amorphous forms of vardenafil for producing the novel dosage forms with controlled release of active ingredient are likewise aspects of the present invention.

Vardenafil and its salts may also be in the form of hydrates in the novel formulation according to the invention. In the context of the invention, hydrates mean compounds comprising water in the crystal. Such compounds may comprise one or more, typically one to six, equivalents of water. Hydrates can be prepared for example by crystallizing the relevant compound from water or a hydrous solvent.

Vardenafil and its salts may also be in the form of solvates in the novel formulation of the present invention. In the context of the invention, solvates mean compounds comprising physiologically tolerated solvents in the crystal.

The following examples serve to illustrate the present invention without limiting it:

### EXAMPLE 1

### Diffusion Pellets

### a) Production of the Active Ingredient-Coated Pellets

| | Composition |
|---|---|
| Starting materials | in mg/20 mg |
| Vardenafil hydrochloride trihydrate | 23.7 |
| micronized | |
| (vardenafil HCl trihydrate micronized)^{a} | |
| Potassium hydrogen tartrate | 40.0 |
| Neutral pellets^{b} | 29.3 |
| HPMC 15 cP | 7.0 |
| (Water)^{c} | (200) |

^{a}Quantity of active ingredient corrresponding to 20 mg of vardenafil
^{b}Sucrose pellets
^{c}Excipient is removed during the process; quantity dependent on the batch size

The neutral pellets are coated with a dispersion consisting of the micronized active ingredient, HPMC, potassium hydrogen tartrate and water in a fluidized bed granulator with Wurster insert.

### b) Coating of the Pellets

| | Composition |
|---|---|
| Starting materials | in mg/20 mg |
| Active ingredient pellets | 100.0 |
| Ethylcellulose^{a} | 6.0 |
| HPMCAS^{b} | 4.0 |
| Triethyl citrate | 2.0 |
| (Water)^{c} | (88.0) |

^{a}Dry matter of Aquacoat ECD 30
^{b}Hydroxypropylmethylcellulose acetate succinate Aqoat AS-LF
^{c}Excipient is removed during the process; quantity dependent on batch size

The active ingredient-loaded pellets are coated by spraying on a dispersion consisting of ethylcellulose dispersion, HPMCAS, TEC and water in a fluidized bed system (with Wurster insert). The coated pellets are subsequently thermally treated at temperatures of 40-90° C. in order to improve the storage stability of the pellet formulation. The coated pellets are then encapsulated.

### EXAMPLES 2 TO 19

### Monolayer Matrix Tablets

| | | | Example 4 |
|---|---|---|---|
| | Example 2 | Example 3 | Quantity |
| Starting materials | Quantity in mg | Quantity in mg | in mg |
| Vardenafil HCl trihydrate | 23.7 | 23.7 | 23.7 |
| micronized^{a} | | | |
| HPMC 100 | 150 | — | — |
| HPMC 400 | — | 150 | — |
| Hydroxypropylcellulose^{b} | — | — | 150 |
| Microcrystalline cellulose | 63.8 | 63.8 | 63.8 |
| Magnesium stearate | 2.5 | 2.5 | 2.5 |

^{a}Quantity of active ingredient corresponding to 20 mg of vardenafil
^{b}Type M

| | Example 5 | Example 6 | Example 7 |
|---|---|---|---|
| | Quantity | Quantity | Quantity |
| Starting materials | in mg | in mg | in mg |
| Vardenafil HCl trihydrate | 23.7 | 23.7 | 23.7 |
| micronized^{a} | | | |
| HPCM 4000 | 130 | — | — |
| HPCM 15000 | — | 90 | — |
| HPCM 100000 | — | — | 80 |
| Microcrystalline cellulose | 59.5 | — | — |
| Lactose^{b} | — | 84.3 | 94.3 |
| Colloidal silicon dioxide | 2.0 | — | — |
| Magnesium stearate | 4.8 | 2.0 | 2.0 |

^{a}Quantity of active ingredient corresponding to 20 mg of vardenafil
^{b}Tablettose 100

### PRODUCTION OF EXAMPLE 2 TO 7

The ingredients of the monolayer matrix tablets with the exception of magnesium stearate and, where appropriate, silicon dioxide are mixed. Magnesium stearate and, where appropriate, silicon dioxide are admixed as admixture. Subsequently, the powder mixture is directly tableted (format: circular 8 mm). The resulting tablets may be coated, for example to ensure photoprotection or for delaying or slowing release.

| | Example 8 | Example 9 | Example 10 |
|---|---|---|---|
| | Quantity | Quantity | Quantity |
| Starting materials | in mg | in mg | in mg |
| Vardenafil HCl trihydrate | 23.7 | 23.7 | 23.7 |
| micronized^{a} | | | |
| Methyacrylic acid copolymer | 100 | — | — |
| Type C^{b} | | | |
| HPMCAS^{c} | — | 100 | — |
| Cellulose acetate phthalate (CAP) | — | — | 100 |
| HPMC 1500 | 123.3 | 123.3 | 123.3 |
| Microcrystalline cellulose | 50.0 | 50.0 | 50.0 |
| Magnesium stearate | 3.0 | 3.0 | 3.0 |

^{a}Quantity of active ingredient corresponding to 20 mg of vardenafil
^{b}Eudragit ® L 100-55
^{c}Hydroxypropylmethylcellulose acetate succinate, Aqoat AS-LF

| | Example 11 | Example 12 | Example 13 |
|---|---|---|---|
| | Quantity | Quantity | Quantity |
| Starting materials | in mg | in mg | in mg |
| Vardenafil HCl trihydrate | 23.7 | 23.7 | 23.7 |
| micronized^{a} | | | |
| Citric acid | 100 | — | — |
| Succinic acid | — | 100 | — |
| Tartaric acid | — | — | 100 |
| HPMC 4000 | 130 | 130 | 130 |
| Microcrystalline cellulose | 59.5 | 59.5 | 59.5 |
| Colloidal silicon dioxide | 2.0 | 2.0 | 2.0 |
| Magnesium stearate | 4.8 | 4.8 | 4.8 |

^{a}Quantity of active ingredient corresponding to 20 mg of vardenafil

| | Example | Example | Example |
|---|---|---|---|
| | 14 | 15 | 16 |
| | Quantity | Quantity | Quantity |
| Starting materials | in mg | in mg | in mg |
| Vardenafil micronized | 20.0 | — | — |
| Vardenafil dihydrate micronized^{a} | — | 21.5 | — |
| Vardenafil dimesylate monohydrate | — | — | 28.6 |
| micronized^{a} | | | |
| HPCM 4000 | 110 | 110 | 115 |
| Tartaric acid | 100 | 100 | 100 |
| Avicel | 63.0 | 63.0 | 79.9 |
| Colloidal silicon dioxide | 2.0 | 1.0 | 2.0 |
| Magnesium stearate | 5.0 | 4.5 | 4.5 |

^{a}Quantity of active ingredient corresponding to 20 mg of vardenafil

| | Example 17 | Example 18 | Example 19 |
|---|---|---|---|
| | Quantity | Quantity | Quantity |
| Starting materials | in mg | in mg | in mg |
| Vardenafil HCl trihydrate | 11.85^{a} | 35.6^{b} | 47.4^{c} |
| micronized^{a} | | | |
| Tartaric acid | 70 | 150 | 150 |
| HPMC 4000 | 100 | 120 | 120 |
| Microcrystalline cellulose | 43.15 | 86.4 | 74.6 |
| Colloidal silicon dioxide | 1.5 | 2.0 | 2.0 |
| Magnesium stearate | 3.5 | 6.0 | 6.0 |

^{a}Quantity of active ingredient corresponding to 10 mg of vardenafil
^{b}Quantity of active ingredient corresponding to 20 mg of vardenafil
^{c}Quantity of active ingredient corresponding to 30 mg of vardenafil

### PRODUCTION OF EXAMPLE 8 TO 19

The ingredients of the monolayer matrix tablets with the exception of magnesium stearate and, where appropriate, silicon dioxide are mixed. Subsequently, the mixture is dry-granulated by roll compaction and, after admixture of silicon dioxide and magnesium stearate, tableted. The resulting tablets can be coated, for example to ensure photoprotection or for delaying or slowing release.

### EXAMPLES 20 TO 25

### 2-Layer Tablets

| | Example 20 | Example 21 | Example 22 |
|---|---|---|---|
| | Quantity | Quantity | Quantity |
| Starting materials | in mg | in mg | in mg |
| Rapid-release layer | | | |
| Vardenafil HCl trihydrate | 11.85 | 11.85 | 11.85 |
| micronized^{a} | | | |
| Microcrystalline cellulose | 105 | 105 | 105 |
| Crospovidone | 6.25 | 6.25 | 6.25 |
| Colloidal silicon dioxide | 0.63 | 0.63 | 0.63 |
| Magnesium stearate | 1.25 | 1.25 | 1.25 |
| Slow-release layer | | | |
| Vardenafil HCl trihydrate | 23.7 | 23.7 | 23.7 |
| micronized^{b} | | | |
| Tartaric acid | 100 | 100 | 100 |
| HPMC 50 | 30 | — | — |
| HPMC 4000 | 30 | 85 | 130.0 |
| Microcrystalline cellulose | 109.8 | 84.8 | 59.5 |
| Colloidal silicon dioxide | 2.0 | 2.0 | 2.0 |
| Magnesium stearate | 4.5 | 4.5 | 4.8 |

^{a}Quantity of active ingredient corresponding to 10 mg of vardenafil
^{b}Quantity of active ingredient corresponding to 20 mg of vardenafil

| | Example 23 | Example 24 | Example 25 |
|---|---|---|---|
| | Quantity | Quantity | Quantity |
| Starting materials | in mg | in mg | in mg |
| Rapid-release layer | | | |
| Vardenafil HCl trihydrate | 5.93 | 5.93 | 5.93 |
| micronized^{a} | | | |
| Microcrystalline cellulose | 68.87 | 59.52 | 59.52 |
| Crospovidone | 4.0 | 3.5 | 3.5 |
| Colloidal silicon dioxide | 0.4 | 0.35 | 0.35 |
| Magnesium stearate | 0.8 | 0.7 | 0.7 |
| Slow-release layer | | | |
| Vardenafil HCl trihydrate | 11.85 | 11.85 | 11.85 |
| micronized^{b} | | | |
| Tartaric acid | 50 | 70 | 90 |
| HPMC 4000 | 100 | 100 | — |
| HPMC 15000 | — | — | 100 |
| Microcrystalline cellulose | 43.15 | 43.15 | 43.15 |
| Colloidal silicon dioxide | 1.5 | 2.0 | 2.0 |
| Magnesium stearate | 3.5 | 4.5 | 4.8 |

^{a}Quantity of active ingredient corresponding to 5 mg of vardenafil
^{b}Quantity of active ingredient corresponding to 10 mg of vardenafil

The ingredients of the rapid-release layer with the exception of the admixture (silicon dioxide, microcrystalline cellulose (about 15% of the total quantity) and magnesium stearate) are mixed and granulated by roll compaction. The components of the slow-release layer with the exception of the admixture components (silicon dioxide and magnesium stearate) are likewise mixed and compacted. After the admixture has been admixed, both granules are tableted in a 2-layer tablet press (format: circular 10 mm for Example 20 to 22 and circular 9 mm for Example 23 to 25). The 2-layer tablets can be coated, for example to ensure photoprotection.

### EXAMPLES 26 TO 31

### Melt Extrudates

| | Example 26 | | |
|---|---|---|---|
| | Quantity | Example 27 | Example 28 |
| Starting materials | in mg | Quantity in mg | Quantity in mg |
| Vardenafil micronized | 20 | 20 | 20 |
| Hydroxypropylcellulose^{a} | 320 | 320 | 320 |
| HPMCAS^{b} | — | — | 50 |
| Maltitol | 40 | — | — |
| Xylitol | — | 40 | 40 |
| Magnesium stearate | 20 | 20 | 20 |

^{a}Type H
^{b}Aqoat AS-LF

| | Example 29 | Example 30 | Example 31 |
|---|---|---|---|
| | Quantity | Quantity | Quantity |
| Starting materials | in mg | in mg | in mg |
| Vardenafil micronized | 20 | 20 | 20 |
| Hydroxypropylcellulose | 320^{a} | 100^{b} | 70^{b} |
| HPMCAS^{c} | 50 | — | — |
| Ammoniomethacrylate | — | 40 | 70 |
| copolymer | | | |
| Type B^{d} | | | |
| Maltitol | 40 | — | — |
| Magnesium stearate | 20 | 10 | 10 |
| Benzoic acid | — | 30 | 30 |

^{a}HPC of type H
^{b}HPC of type M
^{c}Aqoat AS-LF
^{d}Eudragit ® RS PO

The active ingredient is mixed with the excipients for the extrudate. This mixture is extruded at a suitable temperature (e.g. 120-190° C.) in an extruder. Pellets are formed by cutting the extrudate into pieces of suitable length (about 2-3 mm). These melt extrusion pellets can subsequently be rounded off. The pellets can be coated, to prevent adhesion for example during release, with a dispersion consisting for example of an ethyl acrylate methyl methacrylate copolymer dispersion, HPMC, polysorbate, magnesium stearate and water in a fluidized bed granulator with Wurster insert. The extrudates can finally be encapsulated.

### EXAMPLES 32 TO 34

### Osmotic Systems (Bilayer)

| | Example 32 | Example 33 | Example 34 |
|---|---|---|---|
| Starting materials | Quantity in mg | Quantity in mg | Quantity in mg |
| Active ingredient layer | | | |
| Vardenafil micronized | 22.0 | — | — |
| Vardenafil dihydrate | — | 23.7 | — |
| micronized^{a} | | | |
| Vardenafil HCl | — | — | 26.1 |
| trihydrate micronized^{a} | | | |
| HPMC 5 | 5 | 5 | 5 |
| Polyethylene oxide | 101.5 | 99.8 | 97.4 |
| Colloidal silicon | 1.0 | 1.0 | 1.0 |
| dioxide | | | |
| Magnesium stearate | 0.5 | 0.5 | 0.5 |
| Osmotic layer | | | |
| HPMC 5 | 4 | 4 | 4 |
| Sodium chloride | 25 | 25 | 25 |
| Polyethylene oxide | 55 | 55 | 55 |
| Iron oxide red | 0.8 | 0.8 | 0.8 |
| Magnesium stearate | 0.2 | 0.2 | 0.2 |
| Osmosis membrane | | | |
| Cellulose acetate | 28.5 | 28.5 | 28.5 |
| Polyethylene glycol | 1.5 | 1.5 | 1.5 |
| 3350 | | | |

^{a}Quantity of active ingredient corresponding to 22 mg of vardenafil; incl. 10% excess which remains in medicinal form after complete release

2-layer tablets are produced from the components of the active ingredient layer and osmotic layer by dry granulation and tableting (format: circular 8 mm). These tablets are coated with a mixture of cellulose acetate and polyethylene glycol in acetone solution. The tablets are drilled in a suitable way. The tablets can then receive a topcoat, e.g. with a photoprotective coating.

### EXAMPLES 35 TO 37

### Osmotic Systems (Monolayer)

| | Example 35 | Example 36 | Example 37 |
|---|---|---|---|
| Starting materials | Quantity in mg | Quantity in mg | Quantity in mg |
| Tablet | | | |
| Vardenafil micronized | 24.0 | — | — |
| Vardenafil dihydrate | — | 25.8 | — |
| micronized^{a} | | | |
| Vardenafil HCl | — | — | 28.4 |
| trihydrate micronized^{a} | | | |
| Copovidone | 40 | 40 | 40 |
| Xanthan gum | 60 | 60 | 60 |
| Sodium | 15 | 15 | 15 |
| carboxymethylstarch | | | |
| of type A | | | |
| Sodium chloride | 38.5 | 36.7 | 34.1 |
| Sodium bicarbonate | 15 | 15 | 15 |
| HPMC 5 | 5 | 5 | 5 |
| Sodium lauryl sulphate | 0.5 | 0.5 | 0.5 |
| Colloidal silicon | 1.0 | 1.0 | 1.0 |
| dioxide | | | |
| Magnesium stearate | 1.0 | 1.0 | 1.0 |
| Osmosis membrane | | | |
| Cellulose acetate | 13.3 | 13.3 | 13.7 |
| Polyethylene glycol | 0.7 | 0.7 | 1.7 |
| 3350 | | | |

^{a}Quantity of active ingredient corresponding to 24 mg of vardenafil; incl. 20% excess which remains in medicinal form after complete release

Xanthan gum, sodium chloride, sodium bicarbonate and sodium carboxymethylstarch and copovidone are mixed and then granulated with an aqueous dispersion of the active ingredient with HPMC and sodium lauryl sulphate. The granules are mixed with magnesium stearate and colloidal silicon dioxide and tableted (format: circular 8 mm). These tablets are coated with a mixture of cellulose acetate and polyethylene glycol in acetone solution. The tablets are drilled in a suitable way. The tablets can then also receive a topcoat, e.g. with a photoprotective coating.

### EXAMPLE 38

### Release Investigations

The release from two formulations according to the invention (Example 5 and Example 13) is investigated in the paddle apparatus “Apparatus 2” of USP 28-NF 23 (The United States Pharmacopoeia USP 28 2005) at a temperature of 37±0.5° C. and with a stirring speed of 75 rpm using sinkers in media with various pH values. As release media, in each case, 900 ml of 0.1 M hydrochloric acid (pH about 1.1), acetate buffer of pH 4.5 complying with USP (preparation of 1 litre of this buffer: 2.99 g of sodium acetate trihydrate and 14 ml of 2 N acetic acid are dissolved and made up to 1000 ml with demineralized water. If necessary, the pH is adjusted to 4.5±0.05 with sodium hydroxide or 2 N acetic acid) and phosphate buffer of pH 6.8 with 0.1% (m/V) sodium lauryl sulphate (preparation of 1 litre of this medium: 2.747 g of disodium hydrogen orthophosphate dihydrate, 0.475 g of citric acid monohydrate and 10 g of 10% (m/m) sodium lauryl sulphate solution are dissolved and made up to 1000 ml with deionized water. The pH is adjusted to 6.8±0.05 with sodium hydroxide or ortho-phosphoric acid) are used. Samples of the release medium are taken through a filtration unit which must ensure that concomitant substances are removed, and the amount of active ingredient dissolved therein is determined by HPLC with UV-VIS detection. The amount of active ingredient determined in this way is converted into percent by mass of the employed amount of active ingredient.

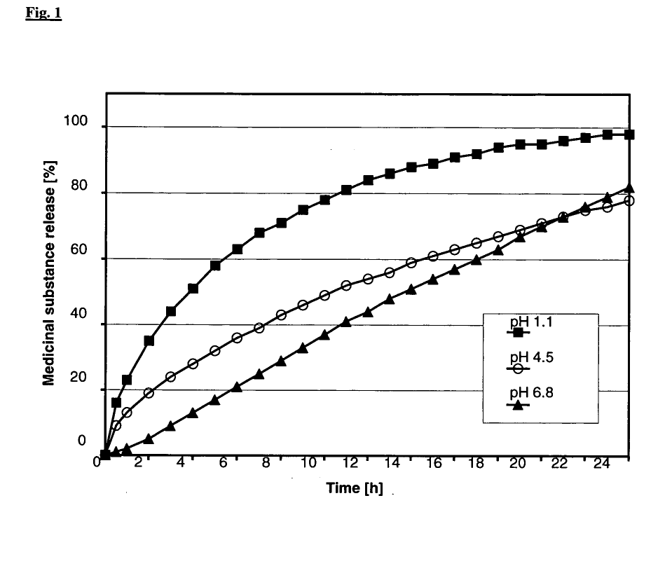

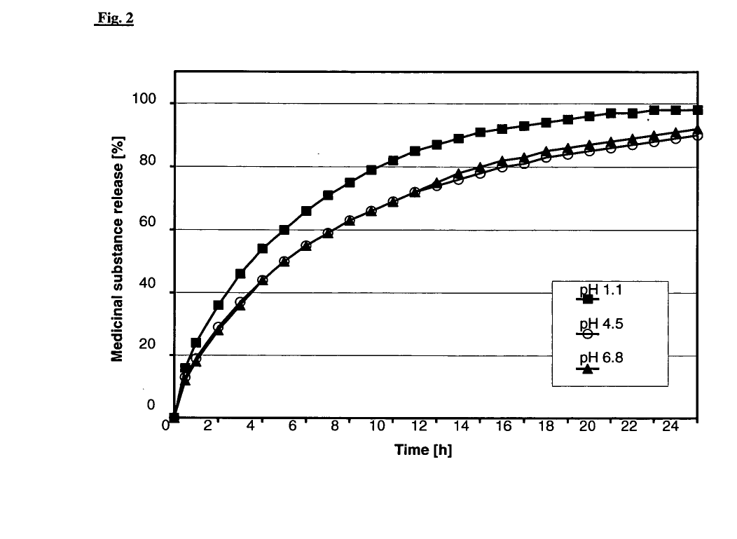

The percentage active ingredient release over time is depicted in FIG. 1 (formulation from Example 5) and FIG. 2 (formulations from Example 13).

The figures show that both formulations are dosage forms with controlled release of active ingredient achieving the above-defined release according to the invention. The formulation of Example 13, whose release is depicted in FIG. 2, represents a preferred embodiment of the present invention because with this dosage form the pH-dependence is distinctly reduced through addition of an acid by comparison with the formulation of Example 5 without pH-modifying additives, whose release is depicted in FIG. 1.

### COMPARATIVE EXAMPLES 1 AND 2

| | Comparative | Comparative |
|---|---|---|
| | Example 1 | Example 2 |
| Starting materials | Quantity in mg | Quantity in mg |
| Vardenafil HCl trihydrate micronized^{a} | 23.7 | 23.7 |
| Hydroxypropylcellulose^{b} | 25 | — |
| HPMC 100000 | — | 150 |
| Tartaric acid | 100 | — |
| Microcrystalline cellulose | 65.8 | 40.8 |
| Colloidal silicon dioxide | 2.0 | 2.0 |
| Magnesium stearate | 3.5 | 3.5 |

^{a}Quantity of active ingredient corresponding to 20 mg of vardenafil
^{b}Type L

### PRODUCTION OF COMPARATIVE EXAMPLES 1 AND 2

The ingredients of the matrix tablets with the exception of silicon dioxide and magnesium stearate are mixed. Silicon dioxide and magnesium stearate are admixed as admixture. The powder mixture are then directly tableted (format: circular 8 mm).

Comparative Examples 1 and 2 are formulations showing an average release rate of 80% in less than 2 hours (Comparative Example 1) and an average release rate of 80% in more than 24 hours (Comparative Example 2). These formulations not according to the invention are unsuitable, in contrast to the dosage forms according to the invention which show an average release rate of between 80% in 2 hours and 80% in 24 hours, for overcoming the problems of the prior art. Thus, the average release rate of Comparative Example 1 is too high to achieve a significant prolongation of the exposure and duration of action compared with prior art formulations. It is furthermore not possible through use of this formulation (Comparative Example 1) to achieve constant blood levels and avoid the occurrence of blood level peaks. By contrast, the average release rate of Comparative Example 2 is too low and leads to a serious loss of bioavailability, so that the blood levels reached are inadequate to achieve the desired clinical effects.

### COMPARATIVE EXAMPLE 3 AND 4

### Pharmacokinetic Parameters of Vardenafil after Oral Administration of a Rapid-Release Tablet Corresponding to the Prior Art

| | Comparative | |
|---|---|---|
| | Example 3 | Comparative Example 4 |
| Parameter | Dose 10 mg | Dose 20 mg |
| AUC [μg * h/L] | 28.8 | 70.0 |
| cₘₐₓ [μg/L] | 7.03 | 18.5 |
| tₘₐₓ [h] | 0.88 | 0.99 |
| Mean residence time [h] | 5.00 | 4.69 |

### EXAMPLE 39 TO 41

### Pharmacokinetic Parameters of Vardenafil after Oral Administration of Formulations According to the Invention with Controlled Release and a Low Initial Release (Dose 20 mg)

| Parameter | Example 39 | Example 40 | Example 41 |
|---|---|---|---|
| AUC [μg * h/L] | 78.8 | 64.4 | 64.5 |
| cₘₐₓ [μg/L] | 7.93 | 5.05 | 4.30 |
| tₘₐₓ [h] | 4.00 | 6.00 | 6.00 |
| Mean residence time [h] | 8.68 | 11.6 | 12.4 |

### EXAMPLE 42 TO 44

### Pharmacokinetic Parameters of Vardenafil after Oral Administration of Formulations According to the Invention with Controlled Release and a High Initial Release (Dose 30 mg)

| Parameter | Example 42 | Example 43 | Example 44 |
|---|---|---|---|
| AUC [μg * h/L] | 119 | 105 | 108 |
| cₘₐₓ [μg/L] | 15.6 | 12.7 | 11.6 |
| tₘₐₓ [h] | 1.5 | 1.0 | 1.0 |
| Mean residence time [h] | 7.7 | 9.4 | 9.9 |

The examples show that the residence time of vardenafil in the body is significantly prolonged with the aid of the dosage forms according to the invention with controlled release of active ingredient by comparison with the residence time resulting on administration of rapid-release formulations corresponding to the prior art (Comparative Example 3 and 4). The kinetic parameters detailed in Examples 39 to 41 for vardenafil after administration of medicament formulations according to the invention with a relatively low initial release show that it is possible with such formulations to achieve not only a prolongation of the MRT but also a distinct reduction in cₘₐₓ compared with the administration of rapid-release formulations of the prior art (Comparative Example 3 and 4). These changes in the pharmacokinetic characteristics MRT and Cₘₐₓ are achieved with a negligible reduction in the AUC, i.e. the bioavailability of the formulations with controlled release of active ingredient and a relatively low initial release is scarcely changed by comparison with a rapid-release formulation. Example 42 to 44 by contrast show the pharmacokinetic data after administration of formulations according to the invention which have a relatively high initial release. It is possible with the aid of these formulations to achieve a rapid rise in the level to reach a desired blood level and additionally to prolong in a relevant manner the mean residence time of the medicinal substance in the body compared with prior art formulations (Comparative Example 3 and 4). It is also true for these formulations that the change in the pharmacokinetic profile is achieved with a virtually (dose-nornalized) unchanged AUC (=bioavailability).

1. Pharmaceutical dosage form with controlled release of active ingredient which comprise the PDE 5 inhibitor vardenafil and/or pharmaceutically acceptable salts and/or hydrates and/or solvates thereof as active ingredient, and which has an average release rate of 5 between 80% in 2 hours and 80% in 24 hours.

2. Pharmaceutical dosage form according to claim 1 with an average release rate of between 80% in 3 hours and 80% in 20 hours.

3. Pharmaceutical dosage form according to claim 1 or 2 with an average release rate of between 80% in 3 hours and 80% in 18 hours and with an initial release of less than 65% of the active ingredient in the first 30 minutes of release.

4. Pharmaceutical dosage form according to at least one of claims 1 to 3, characterized by an initial release of between 0 and 30% of the active ingredient in the first 30 minutes of release.

5. Pharmaceutical dosage form according to at least one of claims 1 to 3, characterized by an initial release of between 30 and 60% of the active ingredient in the first 30 minutes of release.

6. Pharmaceutical dosage form according to at least one of claims 1, 2, 3 or 4, characterized by an average release rate of between 80% in 4 hours and 80% in 18 hours and by an initial release of between 0 and 25% of the active ingredient in the first 30 minutes of release.

7. Pharmaceutical dosage form according to at least one of claims 1, 2, 3 or 5, characterized by an average release rate of between 80% in 3 hours and 80% in 16 hours and by an initial release of between 35 and 60% of the active ingredient in the first 30 minutes of release.

8. Pharmaceutical dosage form according to at least one of claims 1 to 7 for oral use.

9. Pharmaceutical dosage form according to at least one of claims 1 to 8, characterized by a core which comprises the active ingredient and is enveloped by a membrane which controls the release of the active ingredient.

10. Pharmaceutical dosage form according to claim 9, characterized in that the release-controlling membrane comprises a film-forming polymer and a plasticizer.

11. Pharmaceutical dosage form according to claim 9 or 10, characterized in that the release-controlling membrane comprises a film-forming polymer and a pore former.

12. Pharmaceutical dosage form according to at least one of claims 9 to 11, characterized in that it comprises ethylcellulose and/or polymethacrylates as film-forming polymer.

13. Pharmaceutical dosage form according to at least one of claims 9 to 12, characterized in that the active ingredient-containing core comprises a pH-modifying substance.

14. Pharmaceutical dosage form according to at least one of claims 9 to 13, characterized in that the pH-modifying substance is succinic acid, citric acid, tartaric acid or potassium hydrogen tartrate.

15. Pharmaceutical dosage form according to at least one of claims 9 to 14, characterized in that the release-controlling membrane comprises a polymer resistant to gastric juice.

16. Pharmaceutical dosage form according to at least one of claims 1 to 8, characterized by a coated core which comprises one or more swellable excipients which, after penetration in of liquid, cause the coating to split through swelling and volume expansion.

17. Pharmaceutical dosage form according to at least one of claims 1 to 8, characterized in that it comprises the active ingredient in a matrix which delivers the active ingredient through diffusion or erosion.

18. Pharmaceutical dosage form according to claim 17, characterized in that the matrix includes a water-swellable polymer.

19. Pharmaceutical dosage form according to claim 17 or 18, characterized in that it is a tablet.

20. Pharmaceutical dosage form according to at least one of claims 17 to 19, characterized in that the water-swellable polymer is hydroxypropylmethylcellulose or hydroxypropylcellulose.

21. Pharmaceutical dosage form according to at least one of claims 17 to 20, characterized in that the matrix comprises a pH-modifying substance.

22. Pharmaceutical dosage form according to at least one of claims 17 to 21, characterized in that the pH-modifying substance is succinic acid, citric acid or tartaric acid.

23. Pharmaceutical dosage form according to at least one of claims 17 to 22, characterized in that the matrix comprises a polymer resistant to gastric juice.

24. Pharmaceutical dosage form according to at least one of claims 1 to 8 or 17, characterized in that it comprises a melt extrudate of the active ingredient which is produced by incorporating the active ingredient into a matrix by means of a melting process.

25. Pharmaceutical dosage form according to claim 24, characterized in that the melt extrudate includes a thermoplastic polymer.

26. Pharmaceutical dosage form according to claim 24 or 25, characterized in that the melt extrudate comprises a thermoplastic polymer and a plasticizer.

27. Pharmaceutical dosage form according to at least one of claims 24 to 26, characterized in that the thermoplastic polymer is polyvinylpyrrolidone or hydroxypropylcellulose.

28. Pharmaceutical dosage form according to at least one of claims 24 to 27, characterized in that the melt extrudate includes a pH-modifying substance.

29. Pharmaceutical dosage form according to at least one of claims 24 to 28, characterized in that the melt extrudate comprises a polymer resistant to gastric juice.

30. Pharmaceutical dosage form according to at least one of claims 1 to 8, characterized in that it is an osmotic medicinal substance release system.

31. Pharmaceutical dosage form according to claim 30, consisting of:
a core which comprises the active ingredient, where appropriate a hydrophilic polymeric swelling agent and where appropriate a water-soluble substance to initiate osmosis, and where appropriate further pharmaceutically acceptable excipients,
and a shell which consists of a water-permeable material which is impermeable to the components of the active ingredient-containing core, and has at least one orifice through which the ingredients present in the core can be released.

32. Pharmaceutical dosage form according to claim 30 or 31, which comprises polyethylene oxides, xanthan gum and/or copolymers of vinylpyrrolidone and vinyl acetate.

33. Pharmaceutical dosage form according to at least one of claims 1 to 32, which comprises a plurality of identical or different formulation particles as defined in claims 9 to 32.

34. Pharmaceutical dosage form according to at least one of claims 1 to 33, which comprises part of the active ingredient in rapid-release form.

35. Pharmaceutical dosage form according to at least one of claims 1 to 34, which comprises vardenafil and/or vardenafil in the form of its salts, hydrates, solvates, hydrates of the salts and solvates of the salts, and of the polymorphic, crystalline and amorphous forms respectively belonging thereto.

36. Pharmaceutical dosage form according to at least one of claims 1 to 35, which additionally comprises at least one other medicinal substance.

37. Pharmaceutical dosage form according to at least one of claims 1 to 36, which comprises from 1 to 100 mg of the active ingredient calculated as vardenafil.

38. Pharmaceutical dosage form according to at least one of claims 1 to 37, which comprises from 2 to 50 mg of the active ingredient calculated as vardenafil.

39. Pharmaceutical dosage form according to at least one of claims 1 to 38, which includes a matrix which comprises 1 to 30% (m/m) vardenafil hydrochloride trihydrate, 10 to 65% of a water-soluble polymer with a nominal viscosity of at least 50 cP and 10 to 50% (m/m) of an organic acid based on the total mass of the matrix.

40. Pharmaceutical dosage form according to claim 39, which comprises 2 to 20% (m/m) vardenafil hydrochloride trihydrate, 20 to 55% of a water-soluble polymer with a nominal viscosity of at least 50 cP and 20 to 40% (m/m) of an organic acid based on the total mass of the matrix.

41. Pharmaceutical dosage form according to claim 39 or 40 in the form of a multilayer or shell/core tablet which includes a rapid-release layer, a rapid-release shell or rapid-release core.

42. Use of the PDE 5 inhibitor vardenafil and/or of its pharmaceutically acceptable salts and/or hydrates and/or solvates and of the relevant polymorphic, crystalline and amorphous forms for producing a pharmaceutical dosage form as defined in claims 1 to 41.

43. Use of the pharmaceutical dosage forms according to at least one of claims 1 to 41 for the treatment and/or prophylaxis of erectile dysfunction.

44. Use of the pharmaceutical dosage form according to at least one of claims 1 to 41 for the treatment and/or prophylaxis of diseases which experience a therapeutic benefit through increasing the cGMP level.
